(19) United States
(12) Patent Application Publication
(10) Pub. No.: US 2023/0414131 A1
(43) Pub. Date: Dec. 28, 2023

(54) WIRELESS AND RETROFITTABLE IN-SHOE SYSTEM FOR REAL-TIME ESTIMATION OF KINEMATIC AND KINETIC GAIT PARAMETERS

(71) Applicants: THE TRUSTEES OF THE STEVENS INSTITUTE OF TECHNOLOGY, Hoboken, NJ (US); THE TRUSTEES OF COLUMBIA UNIVERSITY IN THE CITY OF NEW YORK, New York, NY (US)

(72) Inventors: Damiano Zanotto, Jersey City, NJ (US); Sunil K. Agrawal, Newark, DE (US); Huanghe Zhang, Jersey City, NJ (US)

(73) Assignees: THE TRUSTEES OF THE STEVENS INSTITUTE OF TECHNOLOGY, Hoboken, NJ (US); THE TRUSTEES OF COLUMBIA UNIVERSITY IN THE CITY OF NEW YORK, New York, NY (US)

(21) Appl. No.: 18/244,847

(22) Filed: Sep. 11, 2023

Related U.S. Application Data
(63) Continuation-in-part of application No. 17/931,527, filed on Sep. 12, 2022.
Application No. 17/931,527 is a continuation of application No. 16/457,730, filed on Jun. 28, 2019, now Pat. No. 11,439,325.
(60) Provisional application No. 63/406,199, filed on Sep. 13, 2022.
(60) Provisional application No. 62/692,568, filed on Jun. 29, 2018.

(51) Int. Cl. A61B 5/11; A61B 5/103; A61B 5/00

(52) U.S. Cl. CPC A61B 5/112; A61B 5/1038; A61B 5/6807; A61B 5/0004; A61B 5/1112; A61B 5/486; A61B 5/7264; A61B 2505/09; A61B 2562/0247; A61B 2562/046

(57) ABSTRACT

A quantitative gait training and/or analysis system includes one or more footwear modules that may include a piezoresistive sensor, an inertial sensor and an independent logic unit. The footwear module functions to permit the extraction of gait kinematics and evaluation thereof in real time, or data may be stored for later reduction and analysis. Embodiments relating to calibration-based estimation of kinematic gait parameters are described, as well as biofeedback embodiments useful in training runners to maintain a time-varying target velocity or pace.

### CROSS-REFERENCE TO RELATED APPLICATIONS

This application is a continuation-in-part of, and claims priority to, pending U.S. patent application Ser. No. 17/931,527, filed on Sep. 12, 2022, which is a continuation of U.S. patent application Ser. No. 16/457,730, filed on Jun. 28, 2019, now U.S. Pat. No. 11,439,325, which patent claims priority to U.S. Provisional Patent Application Ser. No. 62/692,568, filed on Jun. 29, 2018. This application also claims priority to U.S. Provisional Patent Application Ser. No. 63/406,199, filed on Sep. 13, 2022. The contents of the foregoing patent and patent applications are hereby incorporated by reference for all purposes.

### STATEMENT OF GOVERNMENT INTEREST

This invention was made with government support under U.S. government Grant Number CMMI1944203 awarded by the National Science Foundation. The U.S. government may have certain rights in the invention.

### FIELD OF THE INVENTION

The present disclosure relates generally to systems, methods, and devices for gait analysis and training, and, more particularly, to a wearable, autonomous apparatus for quantitative analysis of a subject's gait and/or providing feedback for gait training of the subject. Particular applications of interest arise in sport performance assessment and elderly care.

### BACKGROUND OF THE INVENTION

Pathological gait (e.g., Parkinsonian gait) is clinically characterized using physician observation and camera-based motion-capture systems. Camera-based gait analysis may provide a quantitative picture of gait disorders. However, camera-based motion capture systems are expensive and are not available at many clinics. Auditory and tactile cueing (e.g., metronome beats and tapping of different parts of the body) are often used by physiotherapists to regulate patients' gait and posture. However, this approach requires the practitioner to closely follow the patient and does not allow patients to exercise on their own, outside the laboratory setting.

Compared to traditional laboratory equipment for gait analysis, instrumented footwear systems are more affordable and versatile. These devices can be used to assess the wearers' gait in unrestricted environments, in diverse motor tasks, and over extended time periods.

Quantitative gait analysis is a powerful diagnostic tool for physicians treating patients with gait disorders. Athletic trainers often rely on assessments of the running gait when coaching professional athletes who are recovering from an injury or want to improve their performance. Quantitative gait analysis requires specialized laboratory equipment such as optical motion capture systems and treadmills instrumented with force plates or other force mapping systems. For this reason, the use of gait analysis is currently limited by high operating costs and lack of portability.

In recent years, several instrumented footwear systems have been developed for portable gait assessments. Compared to traditional laboratory equipment, these new systems are more affordable and versatile. However, the amount of parameters these devices can assess is still limited, and their accuracy is usually poor and not comparable to that of standard laboratory equipment.

Correcting form, modifying cadence and foot landing, and training to improve running economy are all significant steps towards improving running performance; however, the current training methods to improve performance, which consist of personal or technology-based coaching, remain either inaccurate or expensive. While off-the-shelf devices are typically limited to interval-based cueing and post-training analysis, the emerging wearable biofeedback systems (WBSs) can provide closed-loop feedback during training. However, most existing WBSs for runners are inaccurate for real-time spatiotemporal gait analysis, limited to temporal gait parameters, or not suitable for out-of-the-lab use.

### OBJECTS OF THE INVENTION

Certain prior art devices are incapable of estimating the user's center of mass (COM) and dynamic margin of stability (MOS). It is therefore an object of the present invention to quantify the position of the COM, the MOS and other indices of dynamic stability. This object is met by the present invention's use of insoles instrumented with inertial, piezoresistive and time-of-flight proximity sensors.

It is another object of the present invention to measure the coordination between upper and lower extremities, as well as to measure a broad set of kinematic and kinetic gait parameters, including, for example, inter-limb parameters such as double support time.

Yet another object of the present invention is to provide wireless functionality and to be lightweight (i.e., below 100 grams) and affordable (i.e., $500 or less), while simultaneously featuring a high sampling rate (500 Hz), making it superior for highly dynamic tasks.

Another object of the present invention is to provide a broad set of information, including plantar pressure maps and center-of-pressure (CoP) trajectories, that can be used for both performance tracking and injury prevention.

Yet another object of the present invention is to make it possible to create remotely-monitored, self-administered walking and balance exercises for the elderly which can potentially increase safety and relieve the financial burden on the healthcare system.

Another object is to provide a completely wireless and portable interface that allows the wearer's own shoes to be retrofitted with the present invention, thereby eliminating the need to modify the shoes themselves.

Yet another object of the invention is to circumvent conventional limitations of portable gait-monitoring systems by presenting novel calibration algorithms based on machine learning and biomechanical models of human locomotion.

A further object of the invention is to enable sport performance evaluation (e.g., running technique) and clinical gait assessments in patients with movement disorders.

Additional objects of the invention include: providing fall risk assessment and fall detection in the elderly, aiding injury prevention in athletes and in the elderly, offering gait or balance rehabilitation with real-time augmented feedback, generating monitoring or activity classification for vulnerable older adults, and aiding pedestrian navigation.

Other objects of the present invention involve the provision of biofeedback features that are useful in training runners, especially long-distance runners, and that allow runners to maintain a targeted pace during training sessions using vibrotactile feedback and/or auditory feedback, which can take the form of continuous music modulation, wherein the parameters (e.g., playback rate, volume and pitch, or the overlay of white noise) of an existing soundtrack are modified on-the-fly (i.e., in real time) responsive to the user's performance during a training session.

### SUMMARY

The present invention is an improvement over and/or a supplement to the systems, devices and methods disclosed U.S. Patent Application Publication No. 2017/0055880, the contents of which are incorporated by reference herein. More particularly, the device of the present invention measures a broad set of spatio-temporal gait parameters (e.g., stride length, foot-ground clearance, foot trajectory, cadence, single and double support times, symmetry ratios and walking speed), as well as kinetic parameters (i.e., dynamic plantar pressure maps, CoP trajectories) during different tasks (e.g., walking and running tasks). By applying custom calibration algorithms (see, for example, FIGS. 1 and 2, which are referenced and described in greater detail hereinbelow) to the raw data measured by the embedded sensors, the device can assess all gait parameters within 1-2% accuracy. This feature allows the present invention to capture subtle changes in gait parameters that are known precursors of injuries or imbalance, and to precisely assess an athlete's running technique.

A system assembled in accordance with the present invention utilizes affordable, mid-level sensors, while providing the option of auditory and vibro-tactile feedback that can be utilized by a user for gait rehabilitation. Another application for the data collected by the system is activity monitoring/classification. This can be realized with machine learning models to automatically classify activities of daily living based on the signals recorded by the system. Additionally, the system can potentially be used with a smartphone equipped with GPS to realize a portable navigation system. Higher accuracy for the system is achieved through the calibration algorithms referenced above and described in greater detail in accompanying FIGS. 1 and 2. Higher accuracy makes it possible to detect subtle changes in the user's gait, which can be precursors of imbalance or injuries.

Most existing portable devices cannot simultaneously estimate temporal parameters, spatial parameters, and kinetic parameters. Although a few such devices may be able to achieve this goal, they suffer from a limited sample rate, which makes them unsuitable for assessments of highly dynamic tasks. Additionally, these devices cannot estimate some important gait parameters, such as foot-ground clearance, foot trajectory, single and double support times, symmetry ratios, CoP trajectories, etc., making them unsuitable for clinical gait assessments.

Traditional gait analysis systems for clinical assessments and sport performance assessments require expensive laboratory equipment, including force plates and optical motion capture systems. Portable gait analysis systems have the advantage of being lightweight and cost-effective, and are not constrained to the laboratory environment, thus making it possible to assess gait metrics in daily-life scenarios. This has important implications for clinical diagnostics, activity monitoring, as well as performance evaluation in sports.

One aspect of the present invention involves the implementation of a novel WBS for continuous music modulation as an effective means to provide accurate, granular, and meaningful auditory feedback to runners, especially long-distance runners, allowing them to maintain a targeted pace during training sessions, especially high intensity interval training (HIIT) sessions.

Another aspect of the present invention involves delivering to a runner vibrotactile feedback, which can be received on the plantar surface of the runner's foot (via insoles) or at the wrist (via a custom-made, wrist-worn device) and which can function as a supplement to, or an alternative to, the aforementioned auditory feedback.

### BRIEF DESCRIPTION OF FIGURES

For a more complete understanding of the present invention, reference is made to the following detailed description of various exemplary embodiments thereof considered in conjunction with the accompanying drawings, in which:

FIG. 1 is a schematic illustration of the first step of a novel two-step calibration approach for the CoP, illustrating a static calibration framework for multi-cell pressure insoles;

FIG. 2 is a schematic illustration of the second step of a novel two-step calibration approach for the CoP, illustrating a dynamic calibration framework for CoP trajectories;

FIG. 3 is an exploded view of an auditory-based and/or vibrotactile-based feedback system for runners, especially long-distance runners, the system being adapted to monitor stride velocity in real time and to function as a cyber-type coach to assist runners in maintaining a targeted pace during high intensity interval training sessions; and

FIG. 4 is a flow diagram of the architecture for the auditory feedback system depicted in FIG. 3.

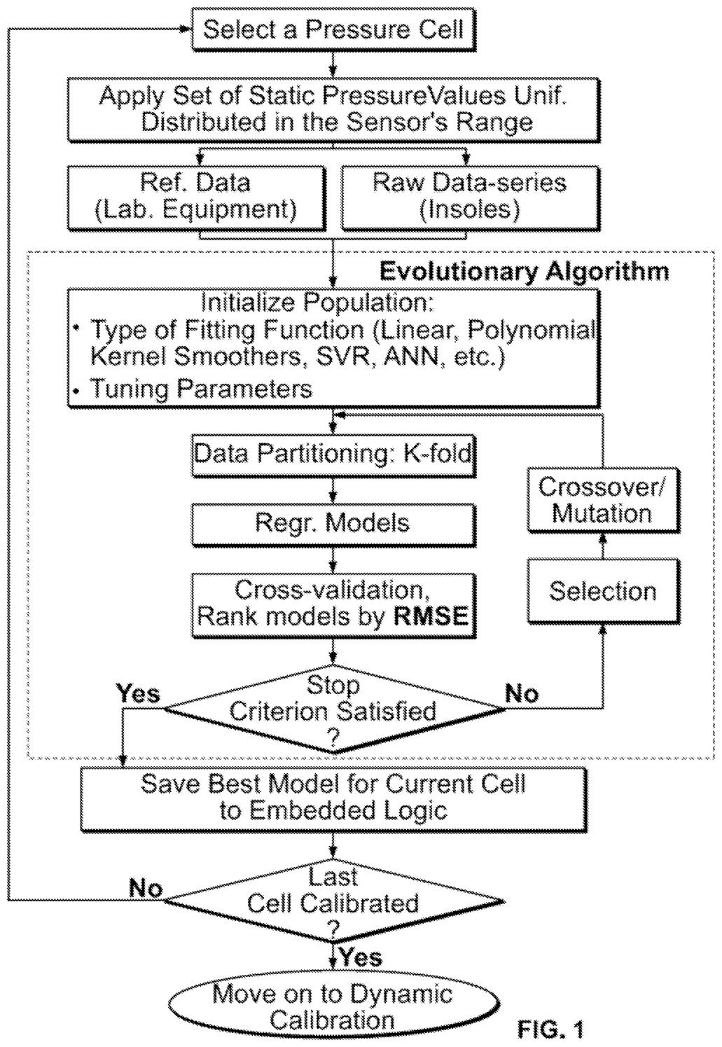

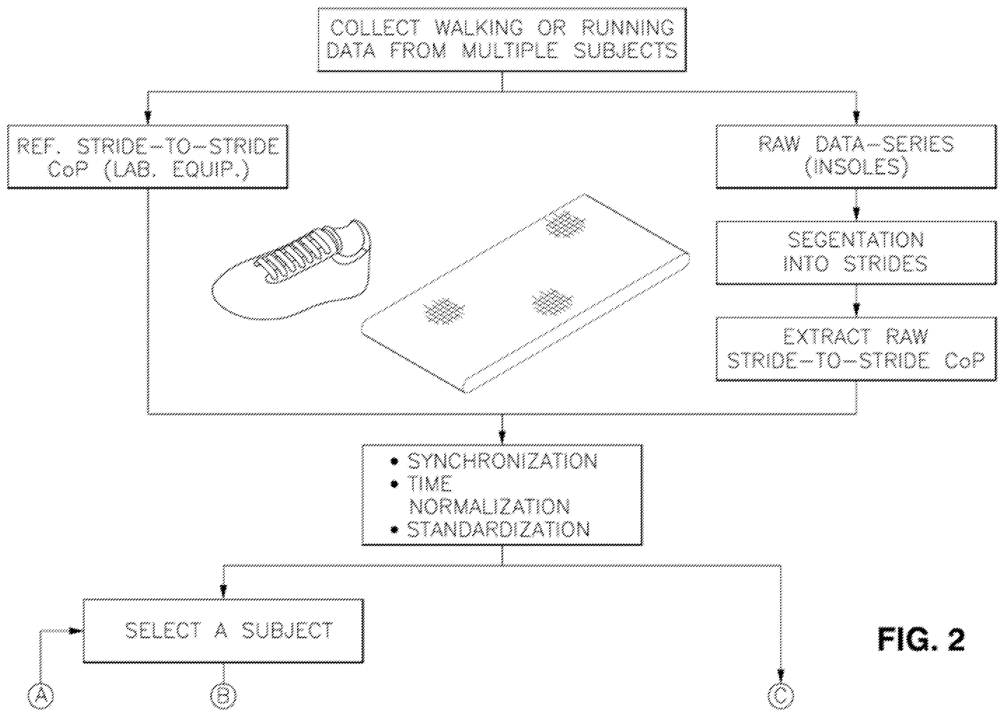

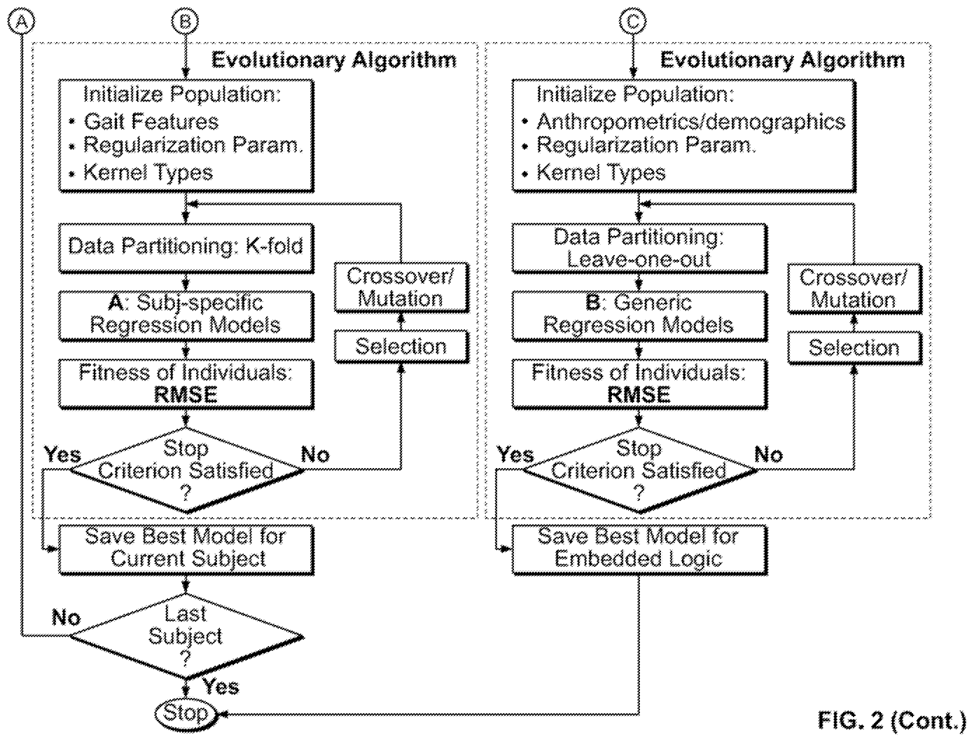

### DETAILED DESCRIPTION OF EXEMPLARY EMBODIMENTS

The following disclosure is presented to provide an illustration of the general principles of the present invention and is not meant to limit, in any way, the inventive concepts contained herein. Moreover, the particular features described in this section can be used in combination with the other described features in each of the multitude of possible permutations and combinations contained herein.

All terms defined herein should be afforded their broadest possible interpretation, including any implied meanings as dictated by a reading of the specification as well as any words that a person having skill in the art and/or a dictionary, treatise, or similar authority would assign thereto.

Further, it should be noted that, as recited herein, the singular forms “a”, “an”, “the”, and “one” include the plural referents unless otherwise stated. Additionally, the terms “comprises” and “comprising” when used herein specify that certain features are present in that embodiment, however, this phrase should not be interpreted to preclude the presence or addition of additional steps, operations, features, components, and/or groups thereof.

All examples and conditional language recited herein are intended for pedagogical purposes to aid the reader in understanding the principles of the invention and the concepts contributed thereby to furthering the relevant art, and are to be construed as being without limitation to such specifically recited examples and conditions. Moreover, all statements herein reciting principles, aspects, and embodiments of the invention, as well as specific examples thereof, are intended to encompass both structural and functional equivalents thereof. Additionally, it is intended that such equivalents include both currently known equivalents as well as equivalents developed in the future, i.e., any elements developed that perform the same function, regardless of structure.

In an embodiment, the present invention is a device comprising two insole modules and a data logger. Each insole module is wireless, having a transmission unit, as well as the ability to accurately measure kinematic and kinetic gait parameters of a user in a variety of dynamic tasks (e.g., walking, running, negotiating stairs, etc.), both outdoor and indoor. In an embodiment, all the data are collected at 500 Hz and sent wirelessly to a battery-powered single-board computer (or mobile device) running a data-logger. In an embodiment, the single-board computer fits inside a running belt that can be worn by the user or can be optionally located offboard within a 30-meter range from the user.

In an embodiment, each insole module consists of an eight-cell piezoresistive sensor, a nine degree-of-freedom inertial sensor, and a custom-made logic unit. The pressure sensors are located, for instance, underneath the calcaneous, the lateral arch, the head of the first, third and fifth metatarsals, the hallux, and the toes, while the inertial sensor is placed, for instance, along the midline of the foot.

In an embodiment, the logic unit includes a microcontroller interfaced with the multi-cell pressure sensor through an eight-channel multiplexer, while communicating with the inertial sensor through a serial connection. In an embodiment, all the data are sampled at 500 Hz and sent through UDP over WLAN to the single-board computer by means of a Wi-Fi module. The logic unit, which can be housed in a plastic enclosure, is powered by, for instance, a small 400 mAh Li-po battery through a step-up voltage regulator.

In an embodiment, the single-board computer runs a Linux distribution with a real-time kernel operating in headless mode. A miniature Wi-Fi router can be connected to the computer, serving as an access point. In use, for example, the computer synchronizes the data incoming from the insole modules and writes them to a micro-SD card. The same data can also be streamed at a lower sample rate (50 Hz) to an easy-to-use user interface running on the user's laptop or mobile phone, whereby the interface allows the user to control the device remotely and to visualize measured data.

One embodiment of the present invention relates to a novel WBS that leverages on-line gait analysis capabilities and continuous music modulation to elicit a target time-varying running speed on the wearer. Specifically it is a novel auditory-based WBS for runners that consists of custom-engineered instrumented insoles, a single-board computer embedded in a running belt, and running earbuds to provide closed-loop auditory feedback to help the wearer adjust his/her running speed to a target pace. This embodiment of the present invention represents a novel WBS capable of accurately estimating stride-by-stride running speed in real-time, while providing intuitive feedback to help the runner to maintain a time-varying target velocity.

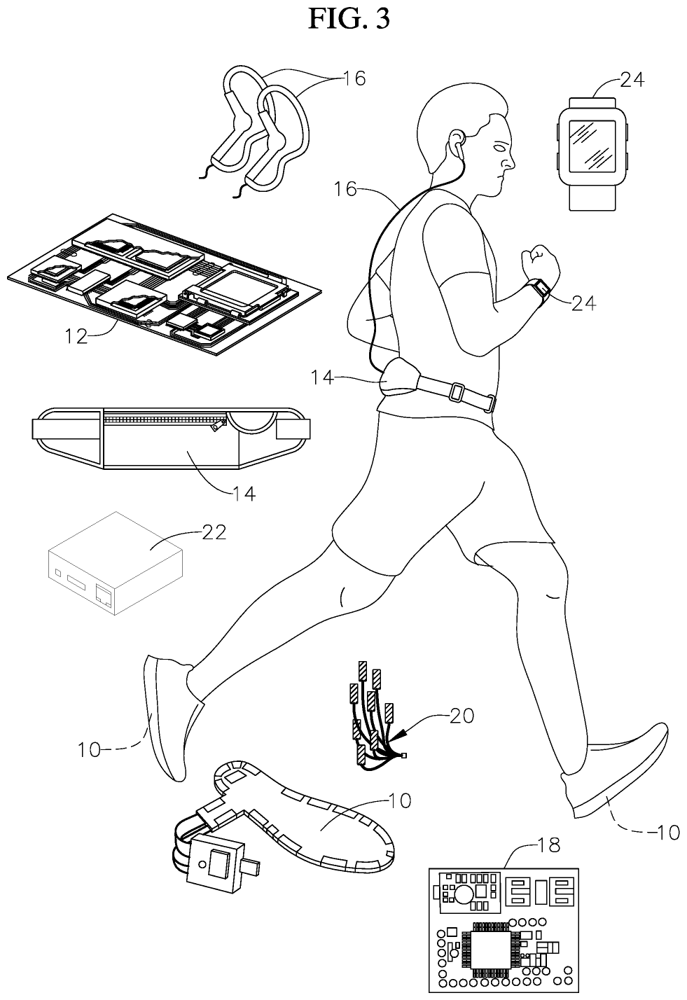

One particular implementation of the foregoing embodiment is depicted in FIG. 3, which shows custom-designed instrumented insoles 10 with shoe-mounted logic units (described below), a Linux single-board computer 12 embedded in a running belt 14, and a pair of running earbuds 16. Related software architecture includes an online gait analysis module, an offline music track selection module, and a closed-loop biofeedback engine with remote control capability through a custom graphical user interface (GUI).

Each instrumented insole is equipped with a 24 g inertial measurement unit 18 (IMU, Yost Labs Inc., OH, US) and an 8-cell array of force sensitive resistors 20 (FSR). The IMU 18 is placed under the medial arch of the foot. The FSR array 20 (IEE S.A., Luxemburg) measures ground reaction forces under the calcaneous, lateral arch, heads of the metatarsals, toes, and hallux. All sensors are pancaked together using anti-abrasion, flexible foam.

The custom-designed logic modules are each mounted on the lateral collar of the user's footwear via plastic clips (not shown). Each logic module is safely enclosed in 3D printed boxes and includes a custom-designed PCB and programmable p-controller (32-bit ARM Cortex-M4, PJRC, OR, USA) powered by a small Li—Po battery. These on-board logic units extract stride-by-stride gait parameters from raw sensors data using the methods described in H. Zhang, D. Zanotto, and S. K. Agrawal, “Estimating CoP trajectories and kinematic gait parameters in walking and running using instrumented insoles,” IEEE Robotics and Automation Letters, vol. 2, no. 4, pp. 2159-2165, 2017, and in H. Zhang, Y. Guo, and D. Zanotto, “Accurate ambulatory gait analysis in walking and running using machine learning models,” IEEE Transactions on Neural Systems and Rehabilitation Engineering, vol. 28, no. 1, pp. 191-202, 2019, both of which publications are incorporated herein by reference. The logic units are also adapted to transmit the resulting metrics to the Linux single-board computer 12 through a UDP network via WLAN, as described below.

The single-board computer 12 can be a 64-bit ARM v8 quad-core CPU (Hardkernel, GyeongGi, South Korea) that fits inside the running belt 14 fashioned on the user's waist. A miniature Wi-Fi router 22 connected to the single-board computer 12 is also embedded in the running belt 14 and serves as an access point for the WBS. The single-board computer 12 serves as a datalogger to store stride-by-stride gait parameters, as well as raw sensor data (333 Hz), while running the algorithms responsible for the auditory feedback modulation, as described below.

The running earbuds 16, which are wired to the single-board computer 12, deliver the auditory stimuli to the user's auditory senses, specifically the user's ears in this embodiment. In an alternative embodiment, the earbuds 16 can be a wireless variant. While the compilation of elements described above may collectively function as a stand-alone, fully-portable system, the Wi-Fi connection allows the user to adjust the biofeedback parameters and enable/disable the device remotely (e.g. by using a laptop).

In use, estimates of stride time (ST) are computed on-line based on FSR signals, from which the timing of initial contacts and toe-off events are also derived. Stride-by-stride estimates of stride length (SL) are also computed on-line, by first removing the contribution of gravity from the accelerometer readings (i.e., by means of orientation estimates obtained with an Extended Kalman Filter), followed by double integration of accelerometric signals with zero-velocity-updates (ZUPT) and velocity drift compensation (VDC), as detailed in S. Minto, D. Zanotto, E. M. Boggs, G. Rosati, and S. K. Agrawal, “Validation of a footwear-based gait analysis system with action-related feedback,” IEEE Transactions on Neural Systems and Rehabilitation Engineering, vol. 24, no. 9, pp. 971-980, 2015, which publication is incorporated herein by reference. At each stride, stride velocity (SV) is determined as the ratio between SL and the corresponding ST. The calculated SV is transmitted to the Linux computer over UDP, for datalogging and for use in the biofeedback engine.

Before a training session takes place, the user's natural running cadence and stride-to-stride variability must be estimated, to match his or her natural rhythms to the tempo of a music track and set an appropriate dead-band for the auditory stimuli. To this end, the user's average natural cadence (CAD) and the standard deviation of his or her stride velocity (SDsv) are estimated offline, after a baseline running session is collected (set to no-feedback mode). CAD is estimated as the dominant frequency of the sum of all FSR signals, restricted to the interval 2-3.5 Hz and converted to steps per minute.

To obtain SDsv, detrended fluctuation analysis (DFA) is applied to the stride-by-stride SV time series and the standard deviation of the resulting detrended series is then calculated. This approach can capture the approximate stride-to-stride variability while filtering out any effect due to local changes in the mean stride. As described below, SDsv determines the maximum velocity errors that are regarded as acceptable during a training exercise.

To mitigate unwanted gait retraining due to conflicting rhythms, and to further personalize the feedback modality, the estimated CAD is used to select a music track that approximately matches the user's (i.e., runner's) rhythm. To this end, a song database sorted by music genre and tempo (beats per minute, BPM) can be developed. The tempo and tempo variability of each song are estimated using beat tracking methods, as described, for instance, in D. P. W. Ellis, “Beat tracking by dynamic programming,” Journal of New Music Research, vol. 36, no. 1, pp. 51-60, 2007, which publication is incorporated herein by reference. Candidate music tracks whose tempo variability exceeds a predefined threshold are automatically excluded from the database. The total number of music tracks included in the final database may exceed 75 songs. A custom Matlab script uses the runner's CAD and favorite music genre as inputs, and outputs a list of music tracks within the chosen music genre, whose tempo is within 10% of the runner's CAD, sorted by lowest to highest absolute percent difference between the runner's CAD and the music tempo. Users can then be asked to choose a song from the list, based on their personal preference.

In an embodiment, the biofeedback engine runs on the Linux single-board computer. It includes of a lower-level software module and a high-level sound synthesis engine. The former is responsible for computing stride-by-stride SV errors and for logging the insole data for off-line processing. When initializing the system, the lower-level module receives the target SV values for the next training session and SDsv as inputs. In use, the user's (i.e., runner's) stride-by-stride SV measured by the insoles is compared with the target speed SVdes to calculate the percent error (OA), which is then sent to the sound synthesis engine through a local UDP socket.

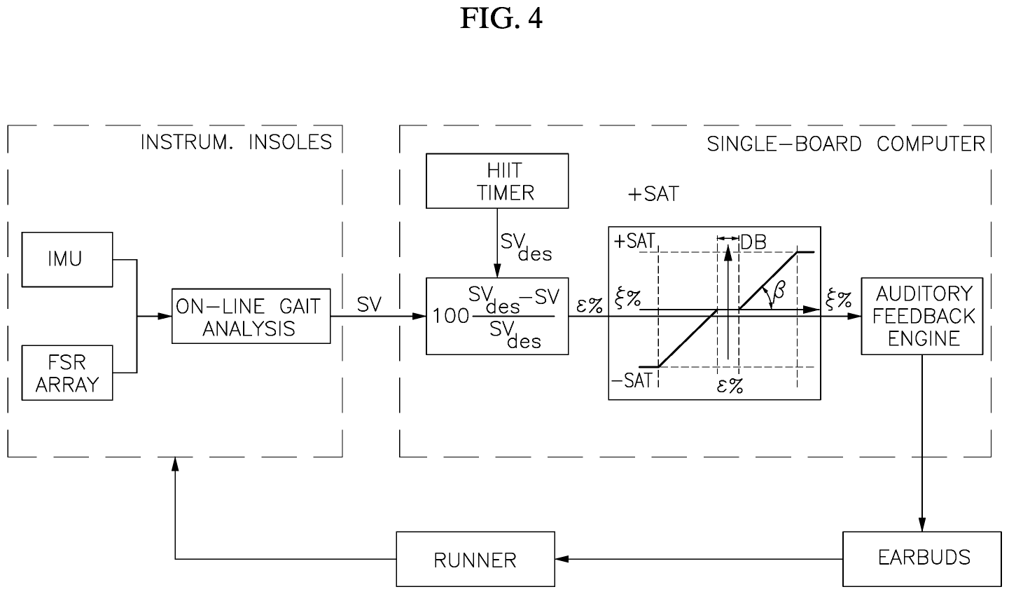

At the higher level, sounds are generated through an open source visual programming language for multimedia as described, for instance, in M. Puckette et al., “Pure data: another integrated computer music environment,” Proceedings of the second intercollege computer music concerts, pp. 37-41, 1996, which publication is incorporated herein by reference. The software can be chosen for its compatibility with ARM-based devices and real-time sound-synthesis capability. The sound synthesis module converts the percent error (ε%) to a corresponding feedback signal (ξ%) according to a linear map with adjustable slope, dead-band, and saturation point (see FIG. 4). In turn, the feedback signal (ξ%) controls the auditory stimuli according to one of the feedback modalities discussed below.

With particular reference to FIG. 4, the system architecture disclosed therein utilizes a HIIT timer to determine the target running speed SVdes ε [SVI, SVh] based on the elapsed time. Stride-by-stride normalized velocity errors (OA) are converted to feedback inputs (ξ%) through a linear map with adjustable slope ß, dead-band (DB) and saturation (SAT) and fed to the auditory feedback engine (i.e., system) that delivers continuous stimuli to the runner through earbuds.

Playback Rate Modulation (PRM): PRM changes the pitch of a music track bidirectionally, trending directly with playback rate. In one embodiment, PRM is achieved by modifying the original sampling rate of a music track (44.1 kHz) on-the-fly, so that a positive feedback signal (ξ%), which indicates that the user is running too fast, results in a corresponding percentage increase of playback rate, and vice versa.

Noise Amplitude Modulation (NAM): NAM is achieved through the overlay of white noise onto a music track. The amplitude of the noise relative to the music track volume is determined by |ξ%|. The sign of the velocity errors is rendered through sound spatialization, whereby a positive (negative) ξ% affects the noise volume delivered to the right (left) ear.

The system described above can be controlled remotely via a Matlab GUI, which allows the user to configure the auditory feedback parameters (volume, music track selection, width of dead-band, saturation point, and slope of the linear mapping), initialize the WBS, and activate the data-logger. The GUI can also enable the user to record the audio heard by the wearer for offline analysis. In an embodiment, a unitary slope can be selected between ε% and ξ% for simplicity. The width of the dead-band can be set to 2 SDsv such that small velocity errors falling within ±1 SDsv will not produce alterations in the auditory stimuli, and the saturation point can be determined empirically through preliminary tests, so that large velocity errors do not result in excessively unpleasant auditory stimuli.

As alluded to above, the present invention can be implemented via a vibrotactile feedback system or modality, instead of or in addition to the auditory feedback system or modality described hereinabove. With reference again to FIG. 3, the vibrotactile feedback system would utilize, for example, a custom-made, wrist-worn device 24 adapted to provide vibrotactile pulse alarms (i.e., stimuli) to the user's (i.e., runner's) wrist based on the gait parameters being measured in accordance with the present invention. In use, the vibrotactile feedback system would generate short vibration bursts, with programmable vibration patterns, to inform the user (i.e., runner) whether his or her current (i.e., real time) velocity is above, below, or at the targeted training velocity. Such vibrotactile cues can be delivered to the user's (i.e., runner's) wrist via the device 24 (see FIG. 3) or via vibrating motors (not shown) in the insoles 10 of the user's (i.e., runner's) shoes.

Other features, attributes and exemplary embodiments of the present invention are disclosed and illustrated in the publication by Huanghe Zhang et al., titled “Estimating CoP Trajectories and Kinematic Gait Parameters in Walking and Running Using Instrumented Insoles,” IEEE Robotics and Automation Letters, Vol. 2, No. 4, October 2017, pp. 2159-2165; in the publication by Huanghe Zhang et al., titled “Regression Models for Estimating Kinematic Gait Parameters with Instrumented Footwear,” IEEE International Conference on Biomedical Robotics and Biomechatronics, August 2018; and in the manuscript entitled “CyberCoach: a Wearable Biofeedback System for Runners” by Michael Gibson et al., all of which publications being incorporated by reference herein in their entireties, and therefore constituting part of the present application.

It will be understood that the embodiments described above, as well those described in the various documents incorporated by reference herein, are merely exemplary and that a person skilled in the art may make many variations and modifications without departing from the spirit and scope of the present invention.

We claim:

1. A biofeedback system for training runners, comprising:
at least one insole module for placement in a shoe of a user, each of said at least one insole module including an array of force-sensitive resistors and an inertial measurement unit;
a logic module communicatively coupled to said array of force-sensitive resistors and to said inertial measurement unit;
a transmission unit;
a computing unit communicatively coupled to said array of force-sensitive resistors and to said inertial measurement unit via said transmission unit, said computing unit being adapted to determine a user's actual running speed in real time and to calculate the difference, if any, between said actual running speed and a targeted running speed; and
feedback means for providing stimuli to a user responsive to said difference, if any, between said actual running speed and said targeted running speed.

2. The biofeedback system of claim 1, wherein said stimuli is in the form of auditory feedback.

3. The biofeedback system of claim 2, wherein auditory feedback is in the form of music.

4. The biofeedback system of claim 3, wherein said music is delivered to a user's audio senses.

5. The biofeedback system of claim 4, wherein said music is selected from a database of songs determined by music genre and tempo.

6. The biofeedback system of claim 5, wherein said music is adapted for continuous modulation.

7. The biofeedback system of claim 6, wherein said music is in the form of an existing soundtrack.

8. The biofeedback system of claim 7, wherein said soundtrack has parameters, including playback rate, volume and pitch.

9. The biofeedback system of claim 8, wherein said parameters are modified in real time in response to a user's performance during a training session.

10. The biofeedback system of claim 9, wherein said tempo is selected in response to a user's average natural cadence.

11. The biofeedback system of claim 6, wherein said modulation is in the form of playback rate modulation.

12. The biofeedback system of claim 6, wherein said modulation is in the form of noise amplitude modulation.

13. The biofeedback system of claim 4, wherein said music is delivered to a user's ears via earbuds.

14. The biofeedback system of claim 1, wherein said stimuli is in the form of vibrotactile feedback.

15. The biofeedback system of claim 14, wherein said vibrotactile feedback is in the form of pulse alarms.

16. The biofeedback system of claim 15, wherein said pulse alarms are delivered to a user's wrist.

17. The biofeedback system of claim 16, wherein said pulse alarms are delivered via a wrist-worn device.

18. The biofeedback system of claim 15, wherein said pulse alarms are delivered to a user via vibrating motors housed in said at least one insole module.

19. A method for calibrating a gait measurement system, comprising the steps of:
i) providing an instrumented insole having a plurality of pressure-sensing cells;
ii) exerting known, uniform pressure on said instrumented insole;
iii) recording a respective output for each of said pressure-sensing cells in response to pressure exerted on said instrumented insole during the performance of step (ii);
iv) applying a plurality of fitting functions to said respective output of each of said pressure-sensing cells, thereby obtaining a plurality of respective model data; and
v) applying cross validation to said respective model data to obtain a calibration model for each of said pressure-sensing cells.

20. A method for calibrating a gait measurement system, comprising the steps of:
i) providing an instrumented insole and a reference measuring apparatus;
ii) recording a first data set from said instrumented insole and a second data set from said reference measuring apparatus;
iii) computing center of pressure trajectories from said first and said second data sets;
iv) validating the accuracy of said center of pressure trajectories using one or more regression models; and
v) calibrating said instrumented insole via said first and said second data sets.
